(19) United States
(12) Patent Application Publication
(10) Pub. No.: US 2015/0284650 A1
(43) Pub. Date: Oct. 8, 2015

(54) N-MONOMETHYL-SUBSTITUTED ANILINES AS COMPONENTS INCREASING THE OXIDATIVE RESISTANCE OF PETROL

(71) Applicant: Alexander Yurievich Frolov, Moscow (RU)

(72) Inventors: Alexander Yurievich Frolov, Moscow (RU); Yuriy Alexandrovich Ivanov, Moscow (RU)

(73) Assignee: ZAKRITOYE AKTSIONERNOE OBSHESTVO, Moscow (RU)

(21) Appl. No.: 14/374,111

(22) PCT Filed: Feb. 21, 2012
(86) PCT No.: PCT/RU2012/000118

§ 371 Date: Jun. 5, 2015

(30) Foreign Application Priority Data
Jan. 25, 2012 (RU) 2012102471

(51) Int. Cl. C10L 1/22; C07C 217/84

(52) U.S. Cl. CPC C10L 1/22; C07C 217/84; C10L 1/223

(57) ABSTRACT

The invention relates to application of N-monomethyl-4-methoxyaniline, N-monomethyl-4-ethoxyaniline and N-monomethyl aniline and their mixtures as oxidation-preventive additives for engine fuel. This invention may be used to improve their performance parameters. It also relates to fuel compositions containing the above-mentioned chemical compounds.

The invention relates to the field of chemistry and chemical technology connected with application of chemical compounds as additives that prevent oxidation of the matters being stabilized by them. Invention may be used for engine fuel stabilization.

The invention relates to chemistry, namely to oxidation-preventive additives to petrol brands; it may be used to improve their performance parameters.

Hydrocarbon fuels produced mainly from oil and gas are subjected to oxidation by known, classic oxidation mechanism inherent to most hydrocarbon compounds, which comprise engine fuels, with certain kinetics of the oxidation process [clauses 1-7 of the List of references]. Hydroperoxides are initial oxidation products; they have low stability and are exposed to further transformations by different directions depending on the hydrocarbon being oxidized and oxidation conditions. At the same time, oxidation rate of hydrocarbons depends upon their molecular weight and structure.

Moreover, the higher molecular weight of the hydrocarbon, the higher it oxidation rate.

During oxidation of petrol brands, which are the mixture of hydrocarbons from different classes with different molecular weight, primary oxidation products may react among themselves. This results to formation of many different oxygen-containing compounds, which may significantly affect the rate and direction of their mutual oxidation. Besides, hydrocarbons, which are not oxidized in their pure state, may oxidize in the presence of unsaturated hydrocarbons. The presence of diene and alkyl aromatic hydrocarbons with double bond in side chains make almost all hydrocarbon mixtures able to oxidation by atmospheric oxygen even at usual temperature.

During oxidation of petrol brands, high-molecular resinous compounds are formed in them as a result of oxidative polymerization and oxidative compensation of primary oxidation products involving both oxygen- and sulfur-containing compounds.

That's why chemical stability of petrol depends also on the presence of such compounds in them.

Oxygen-containing compounds produced from oil or formed during its processing facilitate fuel oxidation thus decreasing its chemical stability. Sulfur-containing (sulfurous) compounds act in the similar way; that's why these compounds are removed preliminarily from raw materials being processed. On this basis, chemical stability of modern petrol brands depends mainly on their hydrocarbon composition and additives introduced in petrol, which have different functional purpose.

It is known that fuels produced on the basis of the thermal cracking petrol may be applied only after addition of special oxidation-preventive agents into them [clause 8 of the List of references].

It is known that the assured shelf life of car petrol brands is at least five years being stored at storage depots located in climatic zones with middle parameters [clause 9 of the List of references].

Alkyl phenols and amine-type compounds are the most effective and widely-used oxidation-preventive additives [clause 10 of the List of references].

It is also known that gum formation considerably decreases in petrol after introduction of oxidation-preventive agents into it. Application of oxidation-preventive agents almost completely solves the problem how to increase chemical stability of petrol produced by thermal and catalytic cracking.

It is known that petrol darkening during its long-term storage is caused, first of all, by oxidation of organosulfur compounds rather than formation of accumulative high-molecular products of hydrocarbon oxidation, which, as a rule, are not intensively colored [clauses 11, 12 of the List of references].

It is known that the petrol oxidation rate increases 1.33 times at the temperature increase by 10° C. despite its composition [clause 13 of the List of references]. Presence of heavy metals, mainly copper and iron, is another important factor during petrol oxidation—these metals act as process catalysts. Moreover, even their insignificant presence accelerates peroxide formation; that's why the search of new anti-oxidants is of current interest.

Thus, oxidation-preventive agent is described in U.S. Pat. No. 7,470,292 as of Dec. 30, 2008, which comprises N,N-di-fluorine-butyl-paraphenilendiamine and Mannich base and is obtained in the reaction between para-nonylphenol with 4-alkylamine and aldehyde in ratio 1:3. However, antioxidants for petrol used nowadays do not always solve performance targets; that's why creation of additives having multi-functional (all-purpose) properties is a forward-looking and economically-advantageous task.

It is known that petrol has to have relevant properties for its effective combustion. These properties are provided by introduction in it a number of additives, each of which has certain functional purpose. Percent content of additives is insignificant in petrol; however, the need in them is still growing owing to increase of petrol production output and consumption. As a rule, existing additives have one functional target and require establishing separate production facilities, which is connected with considerable labor and financial expenditures. That's why creation of additives with multi-functional properties is quite topical and economically-sound decision.

Engineering problem of the invention is to work out and create all-purpose additives for petrol brands, which have all-round properties. It allows applying of only one additive simultaneously for several functional purposes.

Technical result of the invention is to apply octane-increasing additives based on N-monomethyl-4-alcoxy substituted anilines proposed by us as a petrol oxidation prevention [Pat. 2 309 944, the Russian Federation, IPC C07C217/82, C07C235/24, C10L1/223, C10L1/224. Derivatives of Para-Methoxy Anilines Increasing Persistency of Hydrocarbon Fuels to Detonation and Fuel Composition (Alternatives)/Yu. Ivanov, A. Frolov et al. —No 2006111933/04, claimed on Apr. 12, 2006, published on Nov. 10, 2007, Bulletin No 31; Pat. 2 309 943, the Russian Federation, IPC C07C217/82, C07C235/24, C10L1/223, C10L1/224. Application of Para-Ethoxy Aniline Derivatives Increasing Persistency of Hydrocarbon Fuels to Detonation and Fuel Composition (Alternatives)/Yu. Ivanov, A. Frolov et al. —No 2006108236/04, claimed on Mar. 16, 2006, published on Nov. 10, 2007, Bulletin No 31] as antioxidant protection of petrol.

Technical results are achieved by application of octane-increasing additives from the group of N-monomethyl substituted anilines, namely, N-monomethyl-4-methoxy aniline and/or N-monomethyl-4-ethoxy aniline, which are simple mixed ethers, and/or N-monomethyl aniline to prevent petrol against oxidation.

It is known that aromatic amines and phenols with completely shielded imino and phenolic groups are effective anti-oxidants. At the same time, non-shielded or poorly shielded both phenols and imines do not reveal, as a rule, oxidation-preventive activity. However, N-monomethyl substituted anilines, which we have proposed as anti-oxidants for petrol and which are poorly shielded by methyl groups, demonstrated good oxidation-preventive activity while petrol stabilizing against oxidation. These facts are new, unknown and unobvious.

N-monomethyl-4-alkoxy substituted anilines proposed by us, which are simple mixed ethers, efficiently increase petrol withstand to oxidation. That's why, in these circumstances, they are defined as efficient oxidation-preventive agents. Moreover, most substances with well-defined anti-oxidation properties are low toxic as a rule. Besides, the substances proposed by us may facilitate considerable quantitative decrease of formation of polymeric resinous and other products formed after petrol oxidation, which deposit on different parts of internal combustion engines especially on ones being heated. At the same time, etherous groups in additive molecules contain oxygen, which is capable to provide effective and complete petrol combustion and thus to decrease formation of toxic compounds released into the environment as a part of exhaust gases. Thus, application of N-monomethyl-4-alcoxyl substituted anilines proposed by us as petrol components will promote significant increase of petrol quality and, correspondingly, increase of operation life of internal combustion engines and improvement of environmental condition.

The invention is confirmed by the following test results.

Tests were carried out according to GOST R EN ISO 7536-2007, ASTM D-525/IP. Determination of Oxidation Stability. Induction Period Method.

The essence of the test method is to define the time period, within which the tested petrol being in oxygen atmosphere at pressure 700 kPa and temperature 100° C. is able to withstand oxidation.

### Test Results

| | | Induction period, min. | | | | | | | | | | |
|---|---|---|---|---|---|---|---|---|---|---|---|---|
| | | Additive concentration, % wt. | | | | | | | | | | |
| Petrol component name | Additive name | 0 | 0.005 | 0.01 | 0.02 | 0.025 | 0.04 | 0.1 | 0.2 | 0.3 | 0.35 | 3.0 |
| AI-92 commercial petrol | — | 900 | | | | | | | | | | |
| AI-92 commercial petrol | N-monomethyl-4-methoxeaniline | | | | | | | | | | | >3000 |
| AI-92 commercial petrol | N-monomethyl-4-ethoxyaniline | | | | | | | | | | | >3000 |
| Polymer petrol | — | 645 | | | | | | | | | | |
| Polymer petrol | N-monomethyl-4-methoxeaniline | | | | | | | | | | | >3000 |
| Polymer petrol | N-monomethyl-4-ethoxyaniline | | | | | | | | | | | >3000 |
| Catalytic Cracking Petrol (CCP) | — | 215 | | | | | | | | | | |
| — | N-monomethyl-4-methoxeaniline | | | 320 | | | | | | | | |
| — | N-monomethyl-4-ethoxyaniline | | | 322 | | | | | | | | |
| — | N-monomethyl-4-methoxeaniline | | | | 380 | | | | | | | |
| — | N-monomethyl-4-ethoxyaniline | | | | 438 | | | | | | | |
| — | N-monomethyl-4-methoxeaniline | | | | | 406 | | | | | | |
| — | N-monomethyl-4-methoxeaniline | | | | | | | | | | | >3000 |
| — | N-monomethyl aniline | | | 222 | | | | | | | | |
| — | N-monomethyl aniline | | | | 226 | | | | | | | |
| — | N-monomethyl aniline | | | | | | 237 | | | | | |
| — | N-monomethyl aniline | | | | | | | 271 | | | | |
| — | N-monomethyl aniline | | | | | | | | 325 | | | |
| — | N-monomethyl aniline | | | | | | | | | 378 | | |
| — | N-monomethyl aniline | | | | | | | | | | 407 | |
| — | Ionol | | 476 | | | | | | | | | |
| — | Ionol | | | >900 | | | | | | | | |

The similar results were obtained using the fractions of thermal cracking petrol (TCP) and cocking petrol (CP).

While comparing the results of oxidation-protective activity of N-monomethyl aniline and its 4-alkoxy substituted derivatives, which we had proposed as oxidation-preventive agents for petrol, it was stated that imino groups are similarly poorly shielded by methyl substituting group. However, alkoxy groups in 4-alkoxy substituted derivatives, which are in position and act as electron donors, facilitate formation of aminil radicals and simultaneously shield the forth position thereby impeding formation of unstable resonance structures. This structure results in more stable condition of N-monomethyl-4alkoxy substituted derivatives while forming aminil radical. These facts may explain their high oxidation-preventive efficiency as compared with N-monomethyl aniline. Moreover, oxidation-preventive activity of N-monomethyl-4-ethoxy aniline is somewhat higher as compared to N-monomethyl-4-methoxy aniline, probably, due to more efficient shielding of fourth position and electron-donor interaction with monomethyl substituted imino group.

Based on the theoretical concept and obtained experimental data on oxidation-preventive efficiency of N-monomethyl aniline, N-nomomethyl-4-methoxyaniline and N-monomethyl-4-ethoxyaniline in petrol, it follows that N-monomethyl-4-alkoxy substituted anilines, which are simple mixed ethers, considerably surpass N-monomethyl aniline by their efficiency. In this connection, N-monomethyl aniline is of no interest to apply as an oxidation-preventive agent for petrol brands. It is not included separately into the patent claim, just only as a component combined with N-monomethyl-4-alkyl substituted anilines proposed by us.

### LIST OF REFERENCES

1. A. Bakh. Journal of the Russian Physical-Chemical Society, 29, 373/1897/.
2. Engler G., Ber., 80, 660/1897/.
3. N. Semenov. Chain Reactions.—Moscow: Goskhimteorizdat Publisher, 1934.
4. N. Semenov. Concerning Certain Problems of Chemical Kinetics and Reacting Ability.—Moscow: USSR Academy of Sciences, 1958.
5. K. Ivanov. Intermediate Products and Intermediate Reactions of the Hydrocarbon Auto-Oxidation Reaction.—Moscow: Gostoptekhizdat Publisher, 1949.
6. N. Emanuel et al. Chain Reactions of Hydrocarbon Oxidation in Liquid Phase.—Moscow: Nauka Publisher, 1965.
7. N. Chernozhukov, S. Krein. Oxidation Ability of Mineral Oils.—Moscow: Gostoptekhizdat Publisher, 1955.

8. B. Englin, E. Kalantein. Fuel Chemistry and Process Technology.—1956.—Issue 3.—P. 21-31.
9. I. Malysheva et al. Chemistry and Process Technology of Fuels and Oils.—1979.—Issue 7.—P. 18-10. A. Gureev. Application of Car Petrols.—Moscow: Khimiya Publisher, 1972.—368 p.
11. V. Lukyanitsa. Scientific Results Collection of Papers. Chemistry of Oil and Gas.—Issue 2.—Moscow: USSR Academy of Sciences, 1958.—P. 130.
12. E. Karaulova. Scientific Results Collection of Papers. Chemistry of Oil and Gas.—Issue 2.—Moscow: USSR Academy of Sciences, 1958.—P. 130.
13. G. Bloshakov. Restoration and Control over Oil Product Quality.—Leningrad: Nedra Publisher, 1974.—320 p.

1. Application of N-monomethyl-4-methoxyaniline and/or N-monomethyl-4-ethoxyaniline as a component for petroleum prevention against oxidation.

2. Fuel composition containing substances according to claim 1 as an oxidation-preventive agent in amount 0.001-3 wt. %.

3. Fuel composition containing substances according to claim 1 and/or N-monomethyl aniline as an oxidation-preventive agent in amount 0.001-3 wt. %.
